(19) United States
(12) Patent Application Publication
(10) Pub. No.: US 2026/0070949 A1
(43) Pub. Date: Mar. 12, 2026

(54) POLYPEPTIDES HAVING DEAMIDASE INHIBITOR ACTIVITY

(71) Applicant: Novozymes A/S, Bagsvaerd (DK)

(72) Inventors: Marc Dominique MORANT, Bagsvaerd (DK); Morten GJERMANSEN, Greve (DK)

(73) Assignee: Novozymes A/S, Bagsvaerd (DK)

(21) Appl. No.: 18/881,794

(22) PCT Filed: Jul. 3, 2023
(86) PCT No.: PCT/EP2023/068212

§ 371 Date: Jan. 7, 2025

(30) Foreign Application Priority Data
Jul. 15, 2022 (EP) 22185133.0

(51) Int. Cl. C07K 14/195; C12N 9/78

(52) U.S. Cl. CPC C07K 14/195; C12N 9/78; C12Y 305/00

(57) ABSTRACT

The present invention relates to polypeptides having deamidase inhibitor activity and polynucleotides encoding the polypeptides. The invention also relates to nucleic acid constructs, vectors, and host cells comprising the polynucleotides as well as methods of producing and using the polypeptides.

### REFERENCE TO A SEQUENCE LISTING

This application contains a Sequence Listing in computer readable form, which is incorporated herein by reference.

### FIELD OF THE INVENTION

The present invention relates to polypeptides having deamidase inhibitor activity, polynucleotides encoding the polypeptides, nucleic acid constructs, vectors, and host cells comprising the polynucleotides as well as methods of producing and using the polypeptides.

### BACKGROUND

Deamidase enzymes are produced by microbial cells in an inactive proform, which comprises a propeptide domain tightly bound to a deamidase domain. The proform has almost no deamidase activity to protect the viability of the host cell. In nature, the proform is post-processed to remove the propeptide and release the active deamidase outside of the host cell. However, in recombinant expression systems, the propeptide is not naturally removed, and the inactive deamidase proform is secreted outside of the host cell.

The propeptide domain cannot be separated from the deamidase domain by simple cleavage because of the high binding affinity of the propeptide towards the deamidase polypeptide. The binding affinity can be manipulated by mutagenesis to make separation of propeptide and active deamidase easier, but this results in a partly activated intracellular deamidase, which reduces viability of the host cell.

The object of the present invention is to provide new deamidase inhibitors, which can be co-expressed with a deamidase to improve recombinant expression of deamidase by mitigating the negative effect of an active intracellular deamidase on the viability of the host cell.

### SUMMARY OF THE INVENTION

The present invention provides polypeptides capable of inhibiting deamidase activity by reversibly binding to the deamidase and interacting with the deamidase active site.

Accordingly, in a first aspect, the present invention relates to polypeptides having deamidase inhibitor activity selected from the group consisting of:
(a) a polypeptide having at least 60% amino acid sequence identity to SEQ ID NO: 2;
(b) a polypeptide derived from SEQ ID NO: 2 by having 1-30 alterations (e.g., substitutions, deletions and/or insertions) at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a polypeptide derived from the polypeptide of (a) or (b), wherein the N- and/or C-terminal end has been extended by addition of one or more amino acids; and
(d) a fragment of the polypeptide of (a), (b), or (c).

In another aspect, the invention relates to polynucleotides encoding the polypeptides of the present invention; nucleic acid constructs; recombinant expression vectors; recombinant host cells comprising the polynucleotides; and methods of producing the polypeptides.

Other aspects and embodiments of the invention are apparent from the description and examples.

### SEQUENCES

SEQ ID NO: 1: Polynucleotide encoding a deamidase inhibitor from Chryseobacterium sp-62563.

SEQ ID NO: 2: Amino acid sequence of the deamidase inhibitor encoded by SEQ ID NO: 1.

SEQ ID NO: 3: Polynucleotide encoding an active deamidase from Chryseobacterium sp-62563.

SEQ ID NO: 4: Amino acid sequence of the deamidase encoded by SEQ ID NO: 3.

SEQ ID NO: 5: Amino acid sequence motif of deamidase inhibitors.

SEQ ID NO: 6: Amino acid sequence motif of deamidase inhibitors.

SEQ ID NO: 7: Amino acid sequence motif of deamidase inhibitors.

SEQ ID NO: 8: Amino acid sequence motif of deamidase inhibitors.

SEQ ID NO: 9: Amino acid sequence motif of deamidase inhibitors.

SEQ ID NO: 10: Amino acid sequence motif of deamidase inhibitors.

SEQ ID NO: 11: Amino acid sequence motif of deamidase inhibitors.

SEQ ID NO: 12: Amino acid sequence motif of deamidase inhibitors.

SEQ ID NO: 13: Amino acid sequence motif of deamidase inhibitors.

SEQ ID NO: 14: Amino acid sequence motif of deamidase active site.

SEQ ID NO: 15: Amino acid sequence motif of deamidase active site.

SEQ ID NO: 16: Amino acid sequence motif of deamidase active site.

### Definitions

In accordance with this detailed description, the following definitions apply. Note that the singular forms “a,” “an,” and “the” include plural references unless the context clearly dictates otherwise.

Unless defined otherwise or clearly indicated by context, all technical and scientific terms used herein have the same meaning as commonly understood by one of ordinary skill in the art to which this invention belongs.

Deamidase Activity: The term “deamidase activity” means a protein-glutamine glutaminase (also known as glutaminylpeptide glutaminase) activity, as described in EC 3.5.1.44, which catalyzes the hydrolysis of the gamma-amide of glutamine substituted at the carboxyl position or both the alpha-amino and carboxyl positions, e.g., L-glutaminylglycine and L-phenylalanyl-L-glutaminylglycine. Polypeptides having deamidase activity are commonly referred to as deamidases. Thus, deamidases can deamidate glutamine residues in proteins to glutamate residues and are also referred to as protein glutamine deamidase. Deamidases comprise a Cys-His-Asp catalytic triad (e.g., Cys-156, His-197, and Asp-217, as shown in Hashizume et al. “Crystal structures of protein glutaminase and its pro forms converted into enzyme-substrate complex”, Journal of Biological Chemistry, vol. 286, no. 44, pp. 38691-38702) and belong to the InterPro entry IPR041325. In a preferred embodiment, the deamidases of the present invention belong to PFAM domain PF18626. Deamidase amino acid sequences may comprise the amino acid sequence motif DGCYARAH (SEQ ID NO: 14) corresponding to amino acid residues 40-47 of SEQ ID NO: 4, and/or the amino acid sequence motif CYARAH[R/K/Q] (SEQ ID NO: 15) corresponding to amino acid residues 42-48 of SEQ ID NO: 4, and/or the amino acid sequence motif HVA[L/V/I]LVS (SEQ ID NO: 16) corresponding to amino acid residues 83-89 of SEQ ID NO: 4. These motifs overlap with the deamidase active site. Preferably, deamidase activity is the activity exhibited by the polypeptide shown as SEQ ID NO: 4.

Deamidase activity was measured, as described in Example 1, by using a fluorescence substrate comprising a glutamine residue and a fluorescence quenching group. The glutamine residue is converted to a glutamate residue by the deamidase activity, and the substrate is then cleaved by a glutamyl endopeptidase to remove the fluorescence quenching group.

Deamidase activity may also be measured by deamidating a glutamine substrate (for example Cbz-Gln-Gly) and generate ammonia in the process. The ammonia is used as substrate for a glutamate dehydrogenase in combination with α-ketoglutarate to produce glutamate. This latter enzymatic reaction requires NADH as a coenzyme. The depletion of NADH can be followed by kinetic absorbance measurement at 340 nm and is directly proportional to the deamidase activity. The reaction is carried out at pH 7 and 37° C.

Deamidase Inhibitor: The term “deamidase inhibitor” means a sequence of amino acids that interacts with the amino acid residues of the deamidase active site. Thus, deamidase inhibitor activity reduces or inhibits deamidase activity, preferably the deamidase activity exhibited by the polypeptide shown as SEQ ID NO: 4. For example, the deamidase activity can be reduced to less than 90%, less than 80%, less than 70%, less than 60%, less than 50%, or less than 40%, in the presence of the deamidase inhibitor (as compared to the deamidase activity without the presence of the deamidase inhibitor). Polypeptides having deamidase inhibitor activity are commonly referred to as deamidase inhibitors. Deamidase inhibitors may comprise an amino acid sequence motif selected from the group consisting of F[F/Y][I/L/V][F/Q/S][E/K/R]; L[I,T]WY[D,H,K,N]; G[I,M]S[A,P,Q]Q; [D,H,K,N,S][I,L][G,V][I,V][D,E]; [N,H][I,L,M,V,Q][I,V][K,R,Q][E,I,Q]; [D/N][P/S][D/E][H/K/N/Q/R][A/P/S]; and combinations thereof. Deamidase inhibitors may also comprise an amino acid sequence motif selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and combinations thereof.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a polypeptide. The boundaries of the coding sequence are generally determined by an open reading frame, which begins with a start codon, such as ATG, GTG, or TTG, and ends with a stop codon, such as TAA, TAG, or TGA. The coding sequence may be a genomic DNA, cDNA, synthetic DNA, or a combination thereof.

Control sequences: The term “control sequences” means nucleic acid sequences involved in regulation of expression of a polynucleotide in a specific organism or in vitro. Each control sequence may be native (i.e., from the same gene) or heterologous (i.e., from a different gene) to the polynucleotide encoding the polypeptide, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” means any step involved in the production of a polypeptide including, but not limited to, transcription, post-transcriptional modification, translation, post-translational modification, and secretion.

Expression vector: An “expression vector” refers to a linear or circular DNA construct comprising a DNA sequence encoding a polypeptide, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Extension: The term “extension” means an addition of one or more amino acids to the amino and/or carboxyl terminus of a polypeptide, wherein the “extended” polypeptide has deamidase inhibitor activity.

Fragment: The term “fragment” means a polypeptide having one or more amino acids absent from the amino and/or carboxyl terminus of the mature polypeptide, wherein the fragment has deamidase inhibitor activity.

Fusion polypeptide: The term “fusion polypeptide” is a polypeptide in which one polypeptide of the invention is fused at the N-terminus and/or the C-terminus of another polypeptide of the invention. A fusion polypeptide is produced by fusing two or more polynucleotides encoding the polypeptides of the invention together. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12:2575-2583; Dawson et al., 1994, Science 266:776-779). A fusion polypeptide can further comprise a cleavage site between the two polypeptides. Thus, the fusion polypeptides may comprise a cleavage site for a site-specific endopeptidase, for example within 20 amino acids, preferably within 10 amino acids, of the C-terminal end of the first polypeptide. Examples of well-known site-specific endopeptidases include glutamyl endopeptidase (e.g., EC 3.4.21.19 or EC 3.4.21.82), trypsin- and chymotrypsin-like endopeptidases (incl. enteropeptidase). Many other examples of cleavage sites and the corresponding endopeptidases include, but are not limited to, the sites disclosed in Martin et al., 2003, J. Ind. Microbiol. Biotechnol. 3:568-576; Svetina et al., 2000, J. Biotechnol. 76:245-251; Rasmussen-Wilson et al., 1997, Appl. Environ. Microbiol. 63:3488-3493; Ward et al., 1995, Biotechnology 13:498-503; and Contreras et al., 1991, Biotechnology 9:378-381; Eaton et al., 1986, Biochemistry 25:505-512; Collins-Racie et al., 1995, Biotechnology 13:982-987; Carter et al., 1989, Proteins: Structure, Function, and Genetics 6:240-248; and Stevens, 2003, Drug Discovery World 4:35-48.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, of a polypeptide or nucleic acid is not naturally associated with the polypeptide or nucleic acid, i.e., the control sequence is from a gene other than the gene encoding the mature polypeptide.

Host Strain or Host Cell: A “host strain” or “host cell” is an organism into which an expression vector, phage, virus, or other DNA construct, including a polynucleotide encoding a polypeptide of interest (e.g., an amylase) has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

Introduced: The term “introduced” in the context of inserting a nucleic acid sequence into a cell, means “transfection”, “transformation” or “transduction,” as known in the art.

Isolated: The term “isolated” means a polypeptide, nucleic acid, cell, or other specified material or component that has been separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted polypeptide expressed in a host cell.

Mature polypeptide: The term “mature polypeptide” means a polypeptide in its mature form following N-terminal and/or C-terminal processing (e.g., removal of signal peptide). In one aspect, the mature polypeptide is SEQ ID NO: 2.

Mature polypeptide coding sequence: The term “mature polypeptide coding sequence” means a polynucleotide that encodes a mature polypeptide having deamidase inhibitor activity. In one aspect, the mature polypeptide coding sequence is SEQ ID NO: 1.

Native: The term “native” means a nucleic acid or polypeptide naturally occurring in a host cell.

Nucleic acid: The term “nucleic acid” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, and which comprises one or more control sequences operably linked to the nucleic acid sequence.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

Purified: The term “purified” means a nucleic acid, polypeptide or cell that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight or on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

In one aspect, the term “purified” as used herein refers to the polypeptide or cell being essentially free from components (especially insoluble components) from the production organism. In other aspects, the term “purified” refers to the polypeptide being essentially free of insoluble components (especially insoluble components) from the native organism from which it is obtained. In one aspect, the polypeptide is separated from some of the soluble components of the organism and culture medium from which it is recovered. The polypeptide may be purified (i.e., separated) by one or more of the unit operations filtration, precipitation, or chromatography.

Accordingly, the polypeptide may be purified such that only minor amounts of other proteins, in particular, other polypeptides, are present. The term “purified” as used herein may refer to removal of other components, particularly other proteins and most particularly other enzymes present in the cell of origin of the polypeptide. The polypeptide may be “substantially pure”, i.e., free from other components from the organism in which it is produced, e.g., a host organism for recombinantly produced polypeptide. In one aspect, the polypeptide is at least 40% pure by weight of the total polypeptide material present in the preparation. In one aspect, the polypeptide is at least 50%, 60%, 70%, 80% or 90% pure by weight of the total polypeptide material present in the preparation. As used herein, a “substantially pure polypeptide” may denote a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which the polypeptide is natively or recombinantly associated.

It is, therefore, preferred that the substantially pure polypeptide is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total polypeptide material present in the preparation. The polypeptide of the present invention is preferably in a substantially pure form (i.e., the preparation is essentially free of other polypeptide material with which it is natively or recombinantly associated). This can be accomplished, for example by preparing the polypeptide by well-known recombinant methods or by classical purification methods.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, polypeptide or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

Recover: The terms “recover” or “recovery” means the removal of a polypeptide from at least one fermentation broth component selected from the list of a cell, a nucleic acid, or other specified material, e.g., recovery of the polypeptide from the whole fermentation broth, or from the cell-free fermentation broth, by polypeptide crystal harvest, by filtration, e.g. depth filtration (by use of filter aids or packed filter medias, cloth filtration in chamber filters, rotary-drum filtration, drum filtration, rotary vacuum-drum filters, candle filters, horizontal leaf filters or similar, using sheed or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hydro cyclones or similar), or by precipitating the polypeptide and using relevant solid-liquid separation methods to harvest the polypeptide from the broth media by use of classification separation by particle sizes. Recovery encompasses isolation and/or purification of the polypeptide.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the -nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Nucleotides×100)/(Length of Alignment−Total Number of Gaps in Alignment)

Signal Peptide: A “signal peptide” is a sequence of amino acids attached to the N-terminal portion of a protein, which facilitates the secretion of the protein outside the cell. The mature form of an extracellular protein lacks the signal peptide, which is cleaved off during the secretion process.

Subsequence: The term “subsequence” means a polynucleotide having one or more nucleotides absent from the 5′ and/or 3′ end of a mature polypeptide coding sequence; wherein the subsequence encodes a fragment having deamidase inhibitor activity.

Thermal unfolding temperature: The term “thermal unfolding temperature”, also referred to as “melting temperature”, “Tₘ” or “midpoint unfolding temperature”, means a temperature where approximately 50% of the protein is unfolded. Typically, the fraction of folded protein is dominant (>99.999%) at room temperature and decreases as the temperature approaches the melting temperature, Tₘ. At Tₘ, ca. 50% of the molecules are in the folded state, and ca. 50% are in the unfolded state. At temperatures above Tₘ, the unfolded state becomes the dominant species (>50%). A preferred method to determine the thermal unfolding temperature of a deamidase/inhibitor complex with the inhibitor of the invention, is nanoDSF. Using nanoDSF, determination of Tₘ can be carried out at 330 nm, 350 nm, and/or with a ratio of 330 nm/350 nm, wherein Tₘ equals the temperature of the inflection point (IP) of the first derivative. At the inflection point, the first derivative reaches a local maximum or minimum, and the second derivate has an isolated zero. For the present invention, Tₘ or thermal unfolding temperature is the temperature of the inflection point (IP) of the first derivative determined by nanoDSF at 330 nm as described in Example 2.

Other methods for determining the thermal unfolding temperature are a thermal shift assay as described in Current Protocols in Protein Science: “Analysis of protein stability and ligand interactions by thermal shift assay” (K. Huynh and C. L. Partch, 2015) or the methods described in Encyclopedia of Industrial Biotechnology: “Proteins: Thermal Unfolding” (R. Lonescu and L. Shi, 2009).

Thermal unfolding represents an important tool to evaluate protein stability. In order to assess the protein preference to maintain its folded (active) conformation, the protein is exposed to increasing levels of denaturing stress (temperature) and protein stability is determined based on the stress level required to produce a significant fraction of unfolded protein. Factors that affect the thermal stability include, but are not limited to, protein structure, presence of ligands or excipients, binding strength between inhibitor and enzyme, and solvent conditions.

Variant: The term “variant” means a polypeptide having deamidase inhibitor activity comprising a man-made mutation, i.e., a substitution, insertion (including extension), and/or deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### DETAILED DESCRIPTION

### Polypeptides Having Deamidase Inhibitor Activity

The present invention relates to polypeptides having deamidase inhibitor activity selected from the group consisting of:
(a) a polypeptide having at least 60% amino acid sequence identity to SEQ ID NO: 2;
(b) a polypeptide derived from SEQ ID NO: 2 by having 1-30 alterations (e.g., substitutions, deletions and/or insertions) at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a polypeptide derived from the polypeptide of (a) or (b), wherein the N- and/or C-terminal end has been extended by addition of one or more amino acids; and
(d) a fragment of the polypeptide of (a), (b), or (c).

In an aspect, the polypeptide having deamidase inhibitor activity has an amino acid sequence identity of at least 60%, e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% to SEQ ID NO: 2.

The polypeptide preferably comprises, consists essentially of, or consists of the amino acid sequence of SEQ ID NO: 2.

The polypeptide may have an N-terminal and/or C-terminal extension of one or more amino acids, e.g., 1-20 amino acids, 1-10 amino acids, or 1-5 amino acids.

In another aspect, the polypeptide comprises an amino acid sequence motif selected from the group consisting of F[F/Y][I/L/V][F/Q/S][E/K/R]; L[I,T]WY[D,H,K,N]; G[I,M]S[A,P,Q]Q; [D,H,K,N,S][I,L][G,V][I,V][D,E]; [N,H][I,L,M,V,Q][I,V][K,R,Q][E,I,Q]; [D/N][P/S][D/E][H/K/N/Q/R][A/P/S]; and combinations thereof.

In another aspect, the polypeptide comprises an amino acid sequence motif selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and combinations thereof.

In another aspect, the polypeptide is derived from SEQ ID NO: 2 by substitution, deletion or addition of one or more amino acids. In some embodiments, the polypeptide is a variant of SEQ ID NO: 2 comprising a substitution, deletion, and/or insertion at one or more positions. In one aspect, the number of amino acid substitutions, deletions and/or insertions introduced into the polypeptide of SEQ ID NO: 2 is up to 15, e.g., 1, 2, 3, 4, 5, 6, 7, 8, 9, 10, 11, 12, 13, 14, or 15. The amino acid changes may be of a minor nature, that is conservative amino acid substitutions or insertions that do not significantly affect the folding and/or activity of the protein; small deletions, typically of 1-30 amino acids; small amino or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding module.

Essential amino acids in a polypeptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant molecules are tested for deamidase inhibitor activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596:583-589.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, CRISPR gene editing, phage display (e.g., Lowman et al., 1991, Biochemistry 30:10832-10837; U.S. Pat. No. 5,223,409; WO 92/06204), and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a fusion polypeptide.

In an aspect, the polypeptide is isolated.

In another aspect, the polypeptide is purified.

In another aspect, the invention provides a method for . . . .

In yet another aspect, the invention provides a composition comprising a deamidase and a deamidase inhibitor according to the invention.

### Sources of Wildtype Deamidase Inhibitor Polypeptides

Wildtype deamidase inhibitor polypeptides may be obtained from microorganisms (donor strains) of any genus. Preferably, the wildtype deamidase inhibitor polypeptides are obtained from Chryseobacterium species.

For purposes of the present invention, the term “obtained from” as used herein in connection with a given source shall mean that the polypeptide encoded by a polynucleotide is produced by the source or by a strain in which the polynucleotide of the invention has been inserted. In one aspect, the polypeptide obtained from a given source is secreted extracellularly.

In an embodiment, the wildtype deamidase inhibitor polypeptide is obtained from a Chryseobacterium species.

The wildtype deamidase inhibitor polypeptides may be identified and obtained from sources including microorganisms isolated from nature (e.g., soil, composts, water, etc.) or DNA samples obtained directly from natural materials (e.g., soil, composts, water, etc.). Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Davis et al., 2012, Basic Methods in Molecular Biology, Elsevier).

### Polynucleotides

The present invention also relates to polynucleotides encoding a polypeptide of the present invention, as described herein.

The polynucleotide may be mutated by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2:95-107.

In an aspect, the polynucleotide is isolated.

In another aspect, the polynucleotide is purified.

### Nucleic Acid Constructs

The present invention also relates to nucleic acid constructs comprising a polynucleotide of the present invention, wherein the polynucleotide is operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. Techniques for modifying polynucleotides utilizing recombinant DNA methods are well known in the art.

### Promoters

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are described in Sambrook et al., 1989, Molecular Cloning: A Laboratory Manual, Cold Spring Harbor Lab., NY, Davis et al., 2012, supra, and Song et al., 2016, PLOS One 11(7): e0158447.

Examples of suitable promoters for directing transcription of the polynucleotide of the present invention in a filamentous fungal host cell are promoters obtained from Aspergillus, Fusarium, Rhizomucor and Trichoderma cells, such as the promoters described in Mukherjee et al., 2013, “Trichoderma: Biology and Applications”, and by Schmoll and Dattenböck, 2016, “Gene Expression Systems in Fungi: Advancements and Applications”, Fungal Biology.

For expression in a yeast host, examples of useful promoters are described by Smolke et al., 2018, “Synthetic Biology: Parts, Devices and Applications” (Chapter 6: Constitutive and Regulated Promoters in Yeast: How to Design and Make Use of Promoters in S. cerevisiae), and by Schmoll and Dattenböck, 2016, “Gene Expression Systems in Fungi: Advancements and Applications”, Fungal Biology.

### Terminators

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells may be obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells may be obtained from Aspergillus or Trichoderma species, such as obtained from the genes for Aspergillus niger glucoamylase, Trichoderma reesei beta-glucosidase, Trichoderma reesei cellobiohydrolase I, and Trichoderma reesei endoglucanase I, such as the terminators described in Mukherjee et al., 2013, “Trichoderma: Biology and Applications”, and by Schmoll and Dattenböck, 2016, “Gene Expression Systems in Fungi: Advancements and Applications”, Fungal Biology.

Preferred terminators for yeast host cells may be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, Yeast 8:423-488.

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177:3465-3471).

Examples of mRNA stabilizer regions for fungal cells are described in Geisberg et al., 2014, Cell 156(4): 812-824, and in Morozov et al., 2006, Eukaryotic Cell 5(11): 1838-1846.

### Leader Sequences

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Suitable leaders for bacterial host cells are described by Hambraeus et al., 2000, Microbiology 146(12): 3051-3059, and by Kaberdin and Blasi, 2006, FEMS Microbiol. Rev. 30(6): 967-979.

Preferred leaders for filamentous fungal host cells may be obtained from the genes for Aspergillus oryzae TAKA amylase and Aspergillus nidulans triose phosphate isomerase.

Suitable leaders for yeast host cells may be obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

### Polyadenylation Sequences

The control sequence may also be a polyadenylation sequence, a sequence operably linked to the 3′-terminus of the polynucleotide which, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15:5983-5990.

### Signal Peptides

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. Any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Freudl, 2018, Microbial Cell Factories 17:52.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase, such as the signal peptide described by Xu et al., 2018, Biotechnology Letters 40:949-955

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

### Regulatory Sequences

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In fungal systems, these regulatory sequences include the dihydrofolate reductase gene that is amplified in the presence of methotrexate, and the metallothionein genes that are amplified with heavy metals.

### Transcription Factors

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e., by activating the transcription of the gene encoding the protein of interest by binding to its promoter, or indirectly, i.e., by activating the transcription of a further transcription factor which regulates the transcription of the gene encoding the protein of interest, such as by binding to the promoter of the further transcription factor. Suitable transcription factors for fungal host cells are described in WO 2017/144177. Suitable transcription factors for prokaryotic host cells are described in Seshasayee et al., 2011, Subcellular Biochemistry 52:7-23, as well in Balleza et al., 2009, FEMS Microbiol. Rev. 33(1): 133-151.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains at least one element that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous recombination, such as homology-directed repair (HDR), or non-homologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present invention may be inserted into a host cell to increase production of a polypeptide. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

### Host Cells

The present invention also relates to recombinant host cells, comprising a polynucleotide of the present invention operably linked to one or more control sequences that direct the production of a polypeptide of the present invention.

A construct or vector comprising a polynucleotide is introduced into a host cell so that the construct or vector is maintained as a chromosomal integrant or as a self-replicating extrachromosomal vector as described earlier. The choice of a host cell will to a large extent depend upon the gene encoding the polypeptide and its source. The polypeptide can be native or heterologous to the recombinant host cell. Also, at least one of the one or more control sequences can be heterologous to the polynucleotide encoding the polypeptide. The recombinant host cell may comprise a single copy, or at least two copies, e.g., three, four, five, or more copies of the polynucleotide of the present invention.

The host cell may be any microbial cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. In an embodiment, the Bacillus cell is a Bacillus amyloliquefaciens, Bacillus licheniformis and Bacillus subtilis cell.

For purposes of this invention, Bacillus classes/genera/species shall be defined as described in Patel and Gupta, 2020, Int. J. Syst. Evol. Microbiol. 70:406-438.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending, e.g., on the genus. Methods for introducing DNA into prokaryotic host cells are for example described in Heinze et al., 2018, BMC Microbiology 18:56, Burke et al., 2001, Proc. Natl. Acad. Sci. USA 98:6289-6294, Choi et al., 2006, J. Microbiol. Methods 64:391-397, and Donald et al., 2013, J. Bacteriol. 195(11): 2612-2620.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

Fungal cells may be transformed by a process involving protoplast-mediated transformation, Agrobacterium-mediated transformation, electroporation, biolistic method and shock-wave-mediated transformation as reviewed by Li et al., 2017, Microbial Cell Factories 16:168 and procedures described in EP 238023, Yelton et al., 1984, Proc. Natl. Acad. Sci. USA 81:1470-1474, Christensen et al., 1988, Bio/Technology 6:1419-1422, and Lubertozzi and Keasling, 2009, Biotechn. Advances 27:53-75. However, any method known in the art for introducing DNA into a fungal host cell can be used, and the DNA can be introduced as linearized or as circular polynucleotide.

The fungal host cell may be a yeast cell. “Yeast” as used herein includes ascosporogenous yeast (Endomycetales), basidiosporogenous yeast, and yeast belonging to the Fungi Imperfecti (Blastomycetes). For purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell. In a preferred embodiment, the yeast host cell is a Pichia or Komagataella cell, e.g., a Pichia pastoris cell (Komagataella phaffii).

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a preferred embodiment, the filamentous fungal host cell is an Aspergillus, Trichoderma or Fusarium cell. In a further preferred embodiment, the filamentous fungal host cell is an Aspergillus niger, Aspergillus oryzae, Trichoderma reesei, or Fusarium venenatum cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

In an aspect, the host cell is isolated.

In another aspect, the host cell is purified.

In yet another aspect, the host cell further comprises a co-expressed polypeptide exhibiting deamidase activity. In an associated aspect, the invention also relates to methods of producing a polypeptide having deamidase activity, comprising cultivating the recombinant host cell (comprising a co-expressed polypeptide exhibiting deamidase activity) under conditions conducive for production of the polypeptide having deamidase activity; preferably further comprising recovery of the polypeptide having deamidase activity. Such recovery of the polypeptide having deamidase activity may comprise a diafiltration step.

### Methods of Production

The present invention also relates to methods of producing a polypeptide of the present invention, comprising (a) cultivating a recombinant host cell of the present invention under conditions conducive for production of the polypeptide; and optionally, (b) recovering the polypeptide.

The host cell is cultivated in a nutrient medium suitable for production of the polypeptide using methods known in the art. For example, the cell may be cultivated by shake flask cultivation, or small-scale or large-scale fermentation (including continuous, batch, fed-batch, or solid-state, and/or microcarrier-based fermentations) in laboratory or industrial fermentors in a suitable medium and under conditions allowing the polypeptide to be expressed and/or isolated. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptide, including, but not limited to, the use of specific antibodies, formation of an enzyme product, disappearance of an enzyme substrate, or an assay determining the relative or specific activity of the polypeptide.

The polypeptide may be recovered from the medium using methods known in the art, including, but not limited to, collection, centrifugation, filtration, extraction, spray-drying, evaporation, or precipitation. In one aspect, a whole fermentation broth comprising the polypeptide is recovered. In another aspect, a cell-free fermentation broth comprising the polypeptide is recovered.

The polypeptide may be purified by a variety of procedures known in the art to obtain substantially pure polypeptides and/or polypeptide fragments (see, e.g., Wingfield, 2015, Current Protocols in Protein Science; 80(1): 6.1.1-6.1.35; Labrou, 2014, Protein Downstream Processing, 1129:3-10).

In an alternative aspect, the polypeptide is not recovered.

### Solid Formulations

The present invention also relates to enzyme granules/particles comprising a polypeptide of the invention. In an embodiment, the granule comprises a core, and optionally one or more coatings (outer layers) surrounding the core.

The core may have a diameter, measured as equivalent spherical diameter (volume based average particle size), of 20-2000 μm, particularly 50-1500 μm, 100-1500 μm or 250-1200 μm. The core diameter, measured as equivalent spherical diameter, can be determined using laser diffraction, such as using a Malvern Mastersizer and/or the method described under ISO13320 (2020).

In an embodiment, the core comprises a polypeptide having deamidase inhibitor activity of the present invention.

The core may include additional materials such as fillers, fiber materials (cellulose or synthetic fibers), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

The core may include an inert particle with the polypeptide absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA).

The coating may be applied in an amount of at least 0.1% by weight of the core, e.g., at least 0.5%, at least 1%, at least 5%, at least 10%, or at least 15%. The amount may be at most 100%, 70%, 50%, 40% or 30%.

The coating is preferably at least 0.1 μm thick, particularly at least 0.5 μm, at least 1 μm or at least 5 μm. In some embodiments, the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit has few or no uncoated areas. The layer or coating should, in particular, be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

To provide acceptable protection, the salt coating is preferably at least 0.1 μm thick, e.g., at least 0.5 μm, at least 1 μm, at least 2 μm, at least 4 μm, at least 5 μm, or at least 8 μm. In a particular embodiment, the thickness of the salt coating is below 100 μm, such as below 60 μm, or below 40 μm.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular, having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminum. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular, alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710.

Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e., a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄·7H₂O), zinc sulfate heptahydrate (ZnSO₄·7H₂O), sodium phosphate dibasic heptahydrate (Na₂HPO₄·7H₂O), magnesium nitrate hexahydrate (Mg(NO₃)₂(6H₂O)), sodium citrate dihydrate and magnesium acetate tetrahydrate.

Preferably the salt is applied as a solution of the salt, e.g., using a fluid bed.

The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granule may optionally have one or more additional coatings. Examples of suitable coating materials are polyethylene glycol (PEG), methyl hydroxy-propyl cellulose (MHPC) and polyvinyl alcohol (PVA). Examples of enzyme granules with multiple coatings are described in WO 93/07263 and WO 97/23606.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in the Handbook of Powder Technology; Particle size enlargement by C. E. Capes; Vol. 1; 1980; Elsevier. Preparation methods include known feed and granule formulation technologies, e.g.,

(a) Spray dried products, wherein a liquid polypeptide-containing solution is atomized in a spray drying tower to form small droplets which during their way down the drying tower dry to form a polypeptide-containing particulate material. Very small particles can be produced this way (Michael S. Showell (editor); Powdered detergents; Surfactant Science Series; 1998; Vol. 71; pages 140-142; Marcel Dekker).

(b) Layered products, wherein the polypeptide is coated as a layer around a pre-formed inert core particle, wherein a polypeptide-containing solution is atomized, typically in a fluid bed apparatus wherein the pre-formed core particles are fluidized, and the polypeptide-containing solution adheres to the core particles and dries up to leave a layer of dry polypeptide on the surface of the core particle. Particles of a desired size can be obtained this way if a useful core particle of the desired size can be found. This type of product is described in, e.g., WO 97/23606.

(c) Absorbed core particles, wherein rather than coating the polypeptide as a layer around the core, the polypeptide is absorbed onto and/or into the surface of the core. Such a process is described in WO 97/39116.

(d) Extrusion or pelletized products, wherein a polypeptide-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the polypeptide paste, which is harmful to the polypeptide (Michael S. Showell (editor); Powdered detergents; Surfactant Science Series; 1998; Vol. 71; pages 140-142; Marcel Dekker).

(e) Prilled products, wherein a polypeptide-containing powder is suspended in molten wax and the suspension is sprayed, e.g., through a rotating disk atomizer, into a cooling chamber where the droplets quickly solidify (Michael S. Showell (editor); Powdered detergents; Surfactant Science Series; 1998; Vol. 71; pages 140-142; Marcel Dekker). The product obtained is one wherein the polypeptide is uniformly distributed throughout an inert material instead of being concentrated on its surface. U.S. Pat. Nos. 4,016,040 and 4,713,245 describe this technique.

(f) Mixer granulation products, wherein a polypeptide-containing liquid is added to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granulates comprising the polypeptide. Such a process is described in U.S. Pat. No. 4,106,991, EP 170360, EP 304332, EP 304331, WO 90/09440 and WO 90/09428. In a particular aspect of this process, various high-shear mixers can be used as granulators. Granulates consisting of polypeptide, fillers and binders etc. are mixed with cellulose fibers to reinforce the particles to produce a so-called T-granulate. Reinforced particles, are more robust, and release less enzymatic dust.

(g) Size reduction, wherein the cores are produced by milling or crushing of larger particles, pellets, tablets, briquettes etc. containing the polypeptide. The wanted core particle fraction is obtained by sieving the milled or crushed product. Over and undersized particles can be recycled. Size reduction is described in Martin Rhodes (editor); Principles of Powder Technology; 1990; Chapter 10; John Wiley & Sons.

(h) Fluid bed granulation. Fluid bed granulation involves suspending particulates in an air stream and spraying a liquid onto the fluidized particles via nozzles. Particles hit by spray droplets get wetted and become tacky. The tacky particles collide with other particles and adhere to them to form a granule.

(i) The cores may be subjected to drying, such as in a fluid bed drier. Other known methods for drying granules in the feed or enzyme industry can be used by the skilled person. The drying preferably takes place at a product temperature of from 25 to 90° C. For some polypeptides, it is important the cores comprising the polypeptide contain a low amount of water before coating with the salt. If water sensitive polypeptides are coated with a salt before excessive water is removed, the excessive water will be trapped within the core and may affect the activity of the polypeptide negatively. After drying, the cores preferably contain 0.1-10% w/w water.

Non-dusting granulates may be produced, e.g., as disclosed in U.S. Pat. Nos. 4,106,991 and 4,661,452 and may optionally be coated by methods known in the art.

The granulate may further comprise one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of polypeptides by the use of co-granulates is disclosed in WO 2013/188331.

The present invention also relates to protected polypeptides prepared according to the method disclosed in EP 238216.

### Liquid Formulations

The present invention also relates to liquid compositions comprising a polypeptide of the invention. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to, water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials.

In an aspect, the liquid formulation comprises 20-80% w/w of polyol. In one embodiment, the liquid formulation comprises 0.001-2% w/w preservative.

In another embodiment, the invention relates to liquid formulations comprising:
(a) 0.001-25% w/w of a polypeptide having deamidase inhibitor activity of the present invention;
(b) 20-80% w/w of polyol;
(c) optionally 0.001-2% w/w preservative; and
(d) water.

In another embodiment, the invention relates to liquid formulations comprising:
(a) 0.001-25% w/w of a polypeptide having deamidase inhibitor activity of the present invention;
(b) 0.001-2% w/w preservative;
(c) optionally 20-80% w/w of polyol; and
(d) water.

Preferably, the liquid compositions of the above two embodiments further comprises a polypeptide having deamidase activity, for example, in an amount of 0.001-25% w/w.

In another embodiment, the liquid formulation comprises one or more formulating agents, such as a formulating agent selected from the group consisting of polyol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the group consisting of sodium sulfate, dextrin, cellulose, sodium thiosulfate, kaolin and calcium carbonate. In one embodiment, the polyols is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600, more preferably selected from the group consisting of glycerol, sorbitol and propylene glycol (MPG) or any combination thereof.

In another embodiment, the liquid formulation comprises 20-80% polyol (i.e., total amount of polyol), e.g., 25-75% polyol, 30-70% polyol, 35-65% polyol, or 40-60% polyol. In one embodiment, the liquid formulation comprises 20-80% polyol, e.g., 25-75% polyol, 30-70% polyol, 35-65% polyol, or 40-60% polyol, wherein the polyol is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20-80% polyol (i.e., total amount of polyol), e.g., 25-75% polyol, 30-70% polyol, 35-65% polyol, or 40-60% polyol, wherein the polyol is selected from the group consisting of glycerol, sorbitol and propylene glycol (MPG).

In another embodiment, the preservative is selected from the group consisting of sodium sorbate, potassium sorbate, sodium benzoate and potassium benzoate or any combination thereof. In one embodiment, the liquid formulation comprises 0.02-1.5% w/w preservative, e.g., 0.05-1% w/w preservative or 0.1-0.5% w/w preservative. In one embodiment, the liquid formulation comprises 0.001-2% w/w preservative (i.e., total amount of preservative), e.g., 0.02-1.5% w/w preservative, 0.05-1% w/w preservative, or 0.1-0.5% w/w preservative, wherein the preservative is selected from the group consisting of sodium sorbate, potassium sorbate, sodium benzoate and potassium benzoate or any combination thereof.

In another embodiment, the liquid formulation further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof.

### Fermentation Broth Formulations or Cell Compositions

The present invention also relates to a fermentation broth formulation or a cell composition comprising a polypeptide of the present invention. The fermentation broth formulation or the cell composition further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In some embodiments, the fermentation broth formulation or the cell composition comprises a first organic acid component comprising at least one 1-5 carbon organic acid and/or a salt thereof and a second organic acid component comprising at least one 6 or more carbon organic acid and/or a salt thereof. In some embodiments, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In some embodiments, the killed cells and/or cell debris are removed from a cell-killed whole broth to provide a composition that is free of these components.

The fermentation broth formulation or cell composition may further comprise a preservative and/or anti-microbial (e.g., bacteriostatic) agent, including, but not limited to, sorbitol, sodium chloride, potassium sorbate, and others known in the art.

The cell-killed whole broth or cell composition may contain the unfractionated contents of the fermentation materials derived at the end of the fermentation. Typically, the cell-killed whole broth or cell composition contains the spent culture medium and cell debris present after the microbial cells (e.g., filamentous fungal cells) are grown to saturation, incubated under carbon-limiting conditions to allow protein synthesis. In some embodiments, the cell-killed whole broth or cell composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

The invention is further defined by the following numbered embodiments:

Embodiment 1. A polypeptide having deamidase inhibitor activity selected from the group consisting of:
(a) a polypeptide having at least 60% amino acid sequence identity to SEQ ID NO: 2;
(b) a polypeptide derived from SEQ ID NO: 2 by having 1-30 alterations (e.g., substitutions, deletions and/or insertions) at one or more positions;
(c) a polypeptide derived from the polypeptide of (a) or (b), wherein the N- and/or C-terminal end has been extended by addition of one or more amino acids; and
(d) a fragment of the polypeptide of (a), (b), or (c).

Embodiment 2. The polypeptide of embodiment 1, which is derived from SEQ ID NO: 2 by having 1-25 alterations.

Embodiment 3. The polypeptide of embodiment 1, which is derived from SEQ ID NO: 2 by having 1-20 alterations.

Embodiment 4. The polypeptide of embodiment 1, which is derived from SEQ ID NO: 2 by having 1-15 alterations.

Embodiment 5. The polypeptide of embodiment 1, which is derived from SEQ ID NO: 2 by having 1-10 alterations.

Embodiment 6. The polypeptide of embodiment 1, which is derived from SEQ ID NO: 2 by having 1-5 alterations.

Embodiment 7. The polypeptide of any one of the preceding embodiments, wherein the alterations are substitutions, deletions and/or insertions; preferably substitutions.

Embodiment 8. The polypeptide of any one of the preceding embodiments, wherein the alterations are substitutions.

Embodiment 9. The polypeptide of embodiment 1, which has at least 70% amino acid sequence identity to SEQ ID NO: 2.

Embodiment 10. The polypeptide of embodiment 1, which has at least 80% amino acid sequence identity to SEQ ID NO: 2.

Embodiment 11. The polypeptide of embodiment 1, which has at least 90% amino acid sequence identity to SEQ ID NO: 2.

Embodiment 12. The polypeptide of embodiment 1, which has at least 95% amino acid sequence identity to SEQ ID NO: 2.

Embodiment 13. The polypeptide of embodiment 1, which has at least 96%, at least 97%, at least 98%, or at least 99% amino acid sequence identity to SEQ ID NO: 2.

Embodiment 14. The polypeptide of any one of the preceding embodiments, wherein the N- and/or C-terminal end has been extended by addition of 1-20 amino acids.

Embodiment 15. The polypeptide of any one of the preceding embodiments, wherein the N- and/or C-terminal end has been extended by addition of 1-10 amino acids.

Embodiment 16. The polypeptide of any one of the preceding embodiments, wherein the N- and/or C-terminal end has been extended by addition of 1-5 amino acids.

Embodiment 17. The polypeptide of any one of the preceding embodiments, which comprises the amino acid sequence motif F[F/Y][I/L/V][F/Q/S][E/K/R].

Embodiment 18. The polypeptide of any one of the preceding embodiments, which comprises the amino acid sequence motif L[I,T]WY[D,H,K,N].

Embodiment 19. The polypeptide of any one of the preceding embodiments, which comprises the amino acid sequence motif G[I,M]S[A,P,Q]Q.

Embodiment 20. The polypeptide of any one of the preceding embodiments, which comprises the amino acid sequence motif [D,H,K,N,S][I,L][G,V][I,V][D,E].

Embodiment 21. The polypeptide of any one of the preceding embodiments, which comprises the amino acid sequence motif [N,H][I,L,M,V,Q][I,V][K,R,Q][E,I,Q].

Embodiment 22. The polypeptide of any one of the preceding embodiments, which comprises the amino acid sequence motif [D/N][P/S][D/E][H/K/N/Q/R][A/P/S].

Embodiment 23. The polypeptide of any one of the preceding embodiments, which comprises an amino acid sequence motif selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and combinations thereof.

Embodiment 24. The polypeptide of any one of the preceding embodiments, wherein the deamidase inhibitor activity reduces a deamidase activity to less than 90% activity.

Embodiment 25. The polypeptide of any one of the preceding embodiments, wherein the deamidase inhibitor activity reduces a deamidase activity to less than 80% activity.

Embodiment 26. The polypeptide of any one of the preceding embodiments, wherein the deamidase inhibitor activity reduces a deamidase activity to less than 70% activity.

Embodiment 27. The polypeptide of any one of the preceding embodiments, wherein the deamidase inhibitor activity reduces a deamidase activity to less than 60% activity.

Embodiment 28. The polypeptide of any one of the preceding embodiments, wherein the deamidase inhibitor activity reduces a deamidase activity to less than 50% activity.

Embodiment 29. The polypeptide of any one of the preceding embodiments, wherein the deamidase inhibitor activity reduces a deamidase activity to less than 40% activity.

Embodiment 30. The polypeptide of any one of the preceding embodiments, wherein the deamidase activity is derived from a deamidase comprising an amino acid sequence motif selected from the group consisting of SEQ ID NO: 14, SEQ ID NO: 15, SEQ ID NO: 16, and combinations thereof.

Embodiment 31. The polypeptide of any one of the preceding embodiments, wherein the deamidase activity is derived from a polypeptide having deamidase activity derived from a Chryseobacterium species.

Embodiment 32. The polypeptide of any one of the preceding embodiments, wherein the deamidase activity is derived from the polypeptide shown as SEQ ID NO: 4.

Embodiment 33. A polynucleotide encoding the polypeptide of any one of the preceding embodiments.

Embodiment 34. The polynucleotide of embodiment 33, which is isolated and/or purified.

Embodiment 35. A nucleic acid construct or expression vector comprising the polynucleotide of embodiment 33, wherein the polynucleotide is operably linked to one or more control sequences that direct the production of the polypeptide in an expression host.

Embodiment 36. A recombinant host cell comprising the nucleic acid construct or expression vector of embodiment 35.

Embodiment 37. The recombinant host cell of embodiment 36, wherein the polypeptide is heterologous to the recombinant host cell.

Embodiment 38. The recombinant host cell of embodiment 36 or 37, wherein at least one of the one or more control sequences is heterologous to the polynucleotide encoding the polypeptide.

Embodiment 39. The recombinant host cell of any one of embodiments 36-38, which comprises at least two copies, e.g., three, four, or five, or more copies of the polynucleotide of any one of embodiments 33-35.

Embodiment 40. The recombinant host cell of any one of embodiments 36-39, which is a yeast recombinant host cell, e.g., a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

Embodiment 41. The recombinant host cell of any one of embodiments 36-39, which is a filamentous fungal recombinant host cell, e.g., an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell, in particular, an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Embodiment 42. The recombinant host cell of any one of embodiments 36-39, which is a prokaryotic recombinant host cell, e.g., a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, or a Gram-negative bacteria selected from the group consisting of Campylobacter, E. coli, Flavobacterium, Fusobacterium, Helicobacter, Ilyobacter, Neisseria, Pseudomonas, Salmonella, and Ureaplasma cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. Zooepidemicus, Streptomyces achromogenes, Streptomyces avermitilis, Streptomyces coelicolor, Streptomyces griseus, and Streptomyces lividans cells.

Embodiment 43. The recombinant host cell of any one of embodiments 36-39, which is a Bacillus licheniformis cell.

Embodiment 44. The recombinant host cell of any one of embodiments 36-43, which is isolated.

Embodiment 45. The recombinant host cell of any one of embodiments 36-44, which is purified.

Embodiment 46. The recombinant host cell of any one of embodiments 36-45, which further comprises a co-expressed polypeptide exhibiting deamidase activity; preferably the co-expressed polypeptide exhibiting deamidase activity is the polypeptide shown as SEQ ID NO: 4, or a polypeptide having at least 80%, 90%, or 95% amino acid sequence identity to SEQ ID NO: 4.

Embodiment 47. A method of producing the polypeptide of any one of embodiments 1-32, comprising cultivating the recombinant host cell of any one of embodiments 36-45 under conditions conducive for production of the polypeptide.

Embodiment 48. The method of embodiment 47, further comprising recovering the polypeptide.

Embodiment 49. A liquid composition comprising
(a) 0.001-25% w/w of the polypeptide of any one of embodiments 1-32,
(b) polyol, preferably 20-80% w/w of polyol, and
(c) water.

Embodiment 50. The liquid composition of embodiment 49, which further comprises a polypeptide exhibiting deamidase activity.

Embodiment 51. The liquid composition of embodiment 49, which further comprises 0.001-25% w/w of a polypeptide exhibiting deamidase activity.

Embodiment 52. The liquid composition of embodiment 50 or 51, wherein the polypeptide exhibiting deamidase activity has at least 80%, 90%, or 95% amino acid sequence identity to the polypeptide shown as SEQ ID NO: 4.

Embodiment 53. The liquid composition of any of embodiment 50-52, wherein the polypeptide exhibiting deamidase activity is the polypeptide shown as SEQ ID NO: 4.

Embodiment 54. A method of producing a polypeptide having deamidase activity, comprising cultivating the recombinant host cell of embodiment 46 under conditions conducive for production of the polypeptide having deamidase activity.

Embodiment 55. The method of embodiment 54, which further comprises recovering the polypeptide having deamidase activity.

Embodiment 56. The method of embodiment 55, wherein the recovery of the polypeptide having deamidase activity comprises diafiltration.

The present invention is further described by the following examples that should not be construed as limiting the scope of the invention.

### EXAMPLES

### Strains

The Chryseobacterium sp-62563 strain was isolated from a soil sample collected in Sibhult, Sweden in September 2013.

### Example 1

### Deamidase Activity

### Materials

Glutamyl endopeptidase from Bacillus licheniformis
FITC-PP-Dnp (FITC-Ahx-His-His-Gln-Ser-Ser-ED-Dnp) was a custom synthesized substrate molecule from TAG Copenhagen, Kong Georgs Vej 12, DK-2000 Frederiksberg (www.tagc.com), where:
FITC is fluorescein with excitation and emission maxima at approx. 490 nm and 520 nm, Ahx is Aminohexanoic acid,
ED is ethylene diamine, and
Dnp is 2,4-dinitrophenyl (fluorescein fluorescence quencher).

### Deamidase Activity Assay

The assay measures the (relative) activity of active deamidase.

50 μL of deamidase sample was transferred to a standard black 96 well plate and was added:
20 μL of 0.25 μg/mL FITC-PP-Dnp
50 μL of 50 μg/mL glutamyl endopeptidase
130 μL of 100 mM HEPES buffer pH 7.0, and
0.01% v/v Triton X detergent.

A Biotek Synergy H1 fluorescence plate reader was used to measure the fluorescence signal (RFU) for 30 min using emission/excitation wavelengths 485 nm/525 nm.

Data was analyzed for initial rate using variable time intervals depending on the shape of curve (signal vs time). The initial rate for each sample was normalized relative to the initial rate of a fully active deamidase reference enzyme (produced by the wildtype donor strain). The activity was measured as “% initial rate”, i.e., % initial rate of sample molecule relative to initial rate of an active deamidase reference enzyme.

When the deamidase inhibitor of the invention (SEQ ID NO: 2) was added to a sample comprising active deamidase (SEQ ID NO: 4) in an approx. 1:1 ratio (deamidase:inhibitor), the deamidase activity (% initial rate) was significantly reduced.

### Example 2

Nano Differential Scanning Fluorimetry (nanoDSF)—Thermal Unfolding Temperature

Nano differential scanning fluorimetry (nanoDSF) was used to evaluate the conformational stability of the deamidase/inhibitor complex, using the deamidase inhibitor of the invention. The molecules were exposed to a temperature gradient, as indicated below. The resulting changes in structure is reflected in changes in fluorescence intensity and gives a measure of temperature stability. Binding of the deamidase inhibitor to the deamidase contributes to stability of the molecule, thus the nanoDSF thermal unfolding temperatures also give information on the deamidase/inhibitor binding affinity.

His-Tag purified samples were received in an elution buffer from an IMAC (Immobilized Metal Affinity Chromatography) column; 20 mM sodium phosphate; 500 mM sodium chloride; 500 mM imidazole; pH 7.4.

60 μL sample was transferred in replicates to a black-bottomed 384 well plate, and the plate was briefly centrifugated to remove potential air bubbles.

The thermal unfolding temperature of the samples was analyzed using a Prometheus NT.Plex system from NanoTemper Technologies GmbH, with the following settings:
(i) Temperature scan rate: 3.3° C. per minute; and
(ii) Temperature scan interval: 20-95° C.

During the run, samples were loaded in capillaries (Prometheus NT.Plex—Capillary Chips, Standard, Cat #PR-AC002) before being subjected to the temperature gradient. The generated data were analyzed by the PR. Stability analysis v.1.0.1 software. Midpoint unfolding temperatures (Tₘ, ° C.) were annotated based on the first derivative of the 330 nm trace. In some cases, more than one Tₘ may be observed. In these cases, the main peak in the first derivative trace at 330 nm was chosen as the Tₘ of the sample.

### Example 3

### Thermal Unfolding Temperature of Deamidase/Inhibitor Complex

Using the nanoDSF procedure, as described in Example 2, the thermal unfolding temperature of an active deamidase and the corresponding deamidase/inhibitor complex was measured.

TABLE 1
NanoDSF thermal unfolding temperatures of polypeptides.

| | | NanoDSF thermal |
|---|---|---|
| Polypeptides | Sequence | unfolding temperature |
| Active deamidase | SEQ ID NO: 4 | 66.3° C. |
| Active Deamidase | SEQ ID NO: 2 (inhibitor) + | 70.1° C. |
| with added inhibitor | SEQ ID NO: 4 (deamidase) | |

1. A polypeptide having deamidase inhibitor activity selected from the group consisting of:
(a) a polypeptide having at least 60% amino acid sequence identity to SEQ ID NO: 2;
(b) a polypeptide derived from SEQ ID NO: 2 by having 1-30 alterations (e.g., substitutions, deletions and/or insertions) at one or more positions, e.g., 1 or 2 or 3 or 4 or 5 or 6 or 7 or 8 or 9 or 10 or 11 or 12 or 13 or 14 or 15 or 16 or 17 or 18 or 19 or 20 or 21 or 22 or 23 or 24 or 25 or 26 or 27 or 28 or 29 or 30 alterations, in particular substitutions;
(c) a polypeptide derived from the polypeptide of (a) or (b), wherein the N- and/or C-terminal end has been extended by addition of one or more amino acids; and
(d) a fragment of the polypeptide of (a), (b), or (c).

2. The polypeptide of claim 1, comprising an amino acid sequence motif selected from the group consisting of F[F/Y][I/L/V][F/Q/S][E/K/R]; L[I,T]WY[D,H,K,N]; G[I,M]S[A,P,Q]Q; [D,H,K,N,S][I,L][G,V][I,V][D,E]; [N,H][I,L,M,V,Q][I,V][K,R,Q][E,I,Q]; [D/N][P/S][D/E][H/K/N/Q/R][A/P/S]; and combinations thereof.

3. The polypeptide of claim 1, comprising an amino acid sequence motif selected from the group consisting of SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, and combinations thereof.

4. The polypeptide of claim 1, comprising, consisting essentially of, or consisting of SEQ ID NO: 2.

5. The polypeptide of claim 1, wherein the deamidase inhibitor activity reduces a deamidase activity to less than 90% activity.

6. A polynucleotide encoding the polypeptide of claim 1.

7. A nucleic acid construct or expression vector comprising the polynucleotide of claim 6, wherein the polynucleotide is operably linked to one or more control sequences that direct the production of the polypeptide in an expression host.

8. A recombinant host cell comprising the nucleic acid construct or expression vector of claim 7.

9. A method of producing the polypeptide of claim 1, comprising cultivating the recombinant host cell of claim 8 under conditions conducive for production of the polypeptide.

10. A liquid composition comprising
(a) 0.001-25% w/w of the polypeptide of claim 1,
(b) polyol, and
(c) water.

11. The liquid composition of claim 10, which further comprises a polypeptide exhibiting deamidase activity, wherein the polypeptide exhibiting deamidase activity is present in an amount of 0.001-25% w/w.

12. The liquid composition of claim 11, wherein the polypeptide exhibiting deamidase activity is the polypeptide shown as SEQ ID NO: 4, or a polypeptide having at least 80%, 90%, or 95% amino acid sequence identity to SEQ ID NO: 4.

13. The recombinant host cell of claim 8, which further comprises a co-expressed polypeptide exhibiting deamidase activity.

14. A method of producing a polypeptide having deamidase activity, comprising cultivating the recombinant host cell of claim 13 under conditions conducive for production of the polypeptide having deamidase activity.

15. The method of claim 14, which further comprises recovering the polypeptide having deamidase activity.

16. The method of claim 15, wherein the recovery of the polypeptide having deamidase activity comprises diafiltration.

17. The polypeptide of claim 5, wherein deamidase inhibitor activity reduces the deamidase activity of the polypeptide shown as SEQ ID NO: 4 to less than 90%.

18. The recombinant host cell of claim 13, wherein the co-expressed polypeptide exhibiting deamidase activity is the polypeptide shown as SEQ ID NO: 4, or a polypeptide having at least 80%, 90%, or 95% amino acid sequence identity to SEQ ID NO: 4.


SEQUENCE LISTING (16 sequences)

SEQ ID NO: 1 - DNA, 240 bp, Chryseobacterium sp.
  source 1..240: mol_type = genomic DNA
  CDS 1..240: protein_id = 2; translation = QNLTWYNNSTITNITEDPDKPNEVVRVSIGISAQVFYLSKKSTD YKTLLEKATQSFKKSKTYDIGIENKTNIIKEIKEVK
cagaatttaacctggtataacaattccactataaccaatattaccgaagatccggacaaaccgaacgaggttgtacgcgtttctatcggcataagtgctcaggttttttatctttcaaaaaaatctactgactacaaaactcttctggaaaaagcgacccaaagctttaagaaaagtaaaacatatgatataggcattgaaaataaaaccaatatcatcaaagagattaaggaggtcaaa

SEQ ID NO: 2 - protein, 80 aa, Chryseobacterium sp.
QNLTWYNNSTITNITEDPDKPNEVVRVSIGISAQVFYLSKKSTDYKTLLEKATQSFKKSKTYDIGIENKTNIIKEIKEVK

SEQ ID NO: 3 - DNA, 555 bp, Chryseobacterium sp.
  source 1..555: mol_type = genomic DNA
  CDS 1..555: protein_id = 4; translation = AVSVIPNLATLNSLFAQIKNQACGTSTASSPCITFRYPVDGCYA RAHKMRQILLNAGYDCEKQFVYGNLRASTGTCCVSWVYHVAILVSFKNASGVVEKRII DPSLFSSGPVTDAAWRAACTNTSCGSASVSSFANTAGNVYYRSPSGSLLYDNNYVNTN CVLNIFSSLSGCSPSPAPSVASCGF
gcggtaagtgttattcctaacctggccactttaaacagtctgtttgcccaaatcaaaaatcaggcttgcggaacttctacggcatcttcaccatgtatcacattcagatatcctgttgatggctgctatgcaagagctcacaaaatgagacaaatattactgaatgcaggttacgactgtgaaaaacagttcgtatatggtaacctgagagcttctacaggaacttgctgtgtatcttgggtatatcacgtagcaatattggtaagctttaagaatgcttcaggagttgttgagaaaagaatcatagatccttcattattctcaagcggacctgttactgatgctgcatggagagctgcatgtaccaataccagctgtggatctgcttctgtatcttctttcgccaatacggcaggaaatgtttactacagaagtccgtcaggatctttattgtatgataacaattatgtaaataccaactgtgtattgaatatattttcatccctatcaggatgttcaccttctcctgcacctagtgtagctagctgtggattt

SEQ ID NO: 4 - protein, 185 aa, Chryseobacterium sp.
AVSVIPNLATLNSLFAQIKNQACGTSTASSPCITFRYPVDGCYARAHKMRQILLNAGYDCEKQFVYGNLRASTGTCCVSWVYHVAILVSFKNASGVVEKRIIDPSLFSSGPVTDAAWRAACTNTSCGSASVSSFANTAGNVYYRSPSGSLLYDNNYVNTNCVLNIFSSLSGCSPSPAPSVASCGF

SEQ ID NO: 5 - protein, 5 aa, Synthetic construct
FYLSK

SEQ ID NO: 6 - protein, 5 aa, Synthetic construct
FYISK

SEQ ID NO: 7 - protein, 5 aa, Synthetic construct
LTWYN

SEQ ID NO: 8 - protein, 5 aa, Synthetic construct
GISAQ

SEQ ID NO: 9 - protein, 5 aa, Synthetic construct
NIGIE

SEQ ID NO: 10 - protein, 5 aa, Synthetic construct
DIGIE

SEQ ID NO: 11 - protein, 5 aa, Synthetic construct
NIIKE

SEQ ID NO: 12 - protein, 5 aa, Synthetic construct
NIIKQ

SEQ ID NO: 13 - protein, 5 aa, Synthetic construct
DPDKP

SEQ ID NO: 14 - protein, 8 aa, Synthetic construct
DGCYARAH

SEQ ID NO: 15 - protein, 7 aa, Synthetic construct
  VARIANT 7: note = X = R or K or Q
CYARAHX

SEQ ID NO: 16 - protein, 7 aa, Synthetic construct
  VARIANT 4: note = X =L or V or I
HVAXLVS